(19) United States
(12) Patent Application Publication
(10) Pub. No.: US 2024/0002832 A1
(43) Pub. Date: Jan. 4, 2024

(54) A METHOD FOR ISOLATING MOLECULES AND/OR MOLECULAR COMPLEXES

(71) Applicants: CENTRE NATIONAL DE LA RECHERCHE SCIENTIFIQUE, Paris (FR); INSTITUT NATIONAL DES SCIENCES APPLIQUEES DE TOULOUSE, Toulouse (FR)

(72) Inventors: Aline CERF, Saint-Orens de Gameville (FR); Christophe VIEU, Auzeville (FR); Hélène CAYRON, Toulouse (FR); Mouhanad BABI, Hamilton (CA); Denis ESTRADE, Baho (FR); Alexiane LARROCHE, Toulouse (FR)

(73) Assignees: CENTRE NATIONAL DE LA RECHERCHE SCIENTIFIQUE, Paris (FR); INSTITUT NATIONAL DES SCIENCES APPLIQUEES DE TOULOUSE, Toulouse (FR)

(21) Appl. No.: 18/254,446

(22) PCT Filed: Dec. 7, 2021
(86) PCT No.: PCT/EP2021/084657

§ 371 Date: May 25, 2023

(30) Foreign Application Priority Data
Dec. 7, 2020 (EP) 20306512.3

(51) Int. Cl. C12N 15/10; B01L 3/00

(52) U.S. Cl. CPC C12N 15/1006; B01L 3/502761; B01L 2200/0663

(57) ABSTRACT

A method for isolating molecules and/or molecular complexes having a radius of gyration smaller or equal to 2 μm from a complex fluid, including the steps of: a) contacting a complex fluid with a structured capture array having topographical features, wherein the structured capture array is placed in an environment with surrounding humid air humidity of at least 40% based on the maximal moisture content, b) covering the deposited complex fluid with a covering element, wherein the surface tension of the complex fluid between the covering element and the structured capture array defines at least a front and a rear meniscus; and c) dragging either the covering element or the structured capture array in one direction at a speed of at most 2 mm·s⁻¹ for displacing the complex fluid, resulting in that the molecules and/or the molecular complexes are trapped inside the cavities.

The invention relates to the isolation of molecules and/or molecular complexes.

Currently, diagnosis and prognosis of cancers are mostly relied on solid biopsy, i.e. a small portion of the tumour that is taken by a surgical procedure. This biopsy is used for different analysis such as cells state or DNA mutations or. Nevertheless, this technique shows limitations for different reasons and in particular because:
this technique is highly invasive, which implies discomfort for the patient and the requirement to space the puncturing in time,
this technique is limited to visible tumors (not possible on micro-metastasis); some tumors cannot be sampled; puncture limited in space and time (can only be performed once, from one portion of the tumor); implies certain risks for the patient (infections, release in the bloodstream of tumoral material etc), and
the results do not reflect the tumour's genetic heterogeneity since different parts of the tumour accumulate different genetic mutations. When taking a sample on only one area of the tumour, one cannot sequence and detect all the mutations.

Taking the above into account, liquid biopsy appears to be a powerful alternative. Indeed, liquid biopsies are less invasive, can be repeated more often, and could allow the recovery of all the genetic mutations of the tumour. Furthermore, liquid biopsy would enable a more regular follow-up of the entire tumour, and could be a potential tool for early diagnosis. Moreover, potential cancer biomarkers, such as circulating tumor cells (CTCs), miRNAs, circulating DNA (cDNA) and exosomes are located in many biological fluids, such as saliva, urine, blood and its derivatives (plasma and serum).

While saliva is the easiest biological fluid to recover, blood seems to be the richer source and safest choice when the location of the tumour is unknown. Indeed, although 100% of oral cancer patients have cDNA in their saliva and 80% have it in their blood, only 47% to 70% of patients with other types of cancer will be reported with cDNA in saliva.

In blood, circulating DNA can be single-stranded, double-stranded and it can be nuclear, mitochondrial DNA or of viral origin. Free DNA is usually extracted from blood plasma or serum via centrifugation protocols. Extraction from serum yields a greater amount of DNA, but this observation has been potentially attributed to the lysis of white blood cells or other cellular contaminants which then disseminate their DNA into the solution. The extraction protocol may also vary from one study to another, which implies variations in the amount of DNA obtained and its condition. In addition, the time and temperature at which the sample is stored prior to analysis and the duration of the extraction process could also affect the degradation of free genetic material in the sample, or even the release of nucleic acids by other cellular elements.

To optimize the extraction protocol, the blood sample needs to be collected in EDTA-containing tubes, kept cool and handled within 2 hours of collection to minimize contamination, which is highly restrictive.

The aim of the invention is to obviate these drawbacks.

The purpose of the invention is to provide a process for easily isolating molecules and/or molecular complexes.

Thus, the invention relates to a method for, particularly in vitro, isolating molecules and/or molecular complexes having a radius of gyration smaller or equal to 2 μm from a complex fluid, said method comprising the following steps:
a) contacting a complex fluid with a structured capture array having topographical features in the form of a plurality of plane surfaces in-between cavities, wherein the structured capture array is surrounded by humid air,
b) covering the deposited complex fluid with a covering means, wherein the surface tension of the complex fluid between the covering means and the structured capture array defines at least a front and a rear meniscus;
c) dragging either the covering means or the structured capture array in one direction at a speed of at most 2 mm·s⁻¹ for displacing the complex fluid, wherein the front and the rear menisci are displaced on and along the topographical features of said structured capture array toward said direction, wherein the front meniscus covers uncovered topographical features and the rear meniscus uncovers covered topographical features during displacement of the complex fluid, resulting in that:
the molecules and/or the molecular complexes are trapped inside the cavities, and possibly elongated on the plane surfaces toward the direction of the dragging, wherein the humid air has a humidity of at least 40% based on the air maximal humidity.

The process of the invention is advantageously a one-step isolation of low molecular weight markers, such as biomarkers (DNA, exosomes, RNA, proteins), from a whole raw complex fluid. The process of the invention can advantageously perform simultaneous isolation of several (bio)markers based on physical criteria such as the radius of gyration. Isolation can be carried out advantageously on raw complex fluid without the need of any pre-treatment. The invention can be carried out in a wide different field of application from oceanography to healthcare.

By “molecules”, it is meant in the invention a group of two or more atoms held together by covalent bonds. This term encompasses in the invention both natural molecules, as found out in an organism, and synthetic molecules, as produced by the chemical industry.

By “molecular complexes”, it is meant in the invention several molecules bonded together by non-covalent bonds.

The radius of gyration is a dimension that reflects the steric size of an object in a rotational movement. This mechanical concept was generalized to polymer physics, thus making it possible to describe the specific size of a polymer in solution as a function of its total length, its degree of polymerization or its molecular weight. This specific size depends on the molecular interactions along the polymer chain and varies according to the nature of the monomers and the solvent. The radius of gyration of molecules and molecular complexes can be measured by physical methods based on the propagation of electromagnetic waves or neutrons. Examples of such physical methods are given in document D. G. H. Ballard et al, European Polymer Journal, 9, 9, 1973, 965-969.

By complex fluid it is meant in the invention liquid suspensions containing a complex mixture of various elements such as molecules, macromolecules, polymers, cells, particles, aggregates. The complex fluids are non-Newtonian fluids and depart from the classic linear Newtonian relation between stress and shear rate. They exhibit unusual mechanical responses to applied stress or strain due to the geometrical constraints that the phase coexistence imposes. The mechanical response includes transitions between solid-like and fluid-like behaviour as well as fluctuations.

By “simple fluid”, it is meant in the invention a Newtonian fluid for which mechanical behaviour is characterized by a single function of temperature, the viscosity, a measure of the “slipperiness” of the fluid. A stress applied on a simple fluid is directly proportional to the rate of strain.

By “humidity”, it is meant in the invention the ratio (in percentage) of the partial pressure of water vapor to the air maximal humidity. The air maximal humidity corresponds to the equilibrium vapor pressure of water at a given temperature. Humidity can be measured by hygrometers. Air maximal humidity can be estimated by several empirical formulas known in the art. The commonly used formula is the Arden Buck equation.

Step a)

The aim of the first step of the process according to the invention is to put into contact the complex fluid, that contains the molecules and/or molecular complexes to be isolated, with a capture array having cavities in which the said molecules and/or molecular complexes will be isolated.

This simple contact is not sufficient to trigger the isolation of the molecules and/or molecular complexes into the cavities of the capture array. Indeed, the probability of self-isolation of the molecules and/or molecular complexes is very low. In particular, because of that the complex fluid, by nature, contains a huge variety of movement inside the complex fluid which drive the molecules and molecular complexes in all directions in a non-predictable manner, and make it hard, even impede, their isolation on the capture array.

The process of the invention is carried out in an environment with a humid surrounding air. The humidity of the surrounding air is a crucial parameter for the isolation of the molecules and/or molecular complexes. Indeed, if the surrounding air is not sufficiently humid, i.e. has not a humidity of at least 40% based on the maximal moisture content of the air, the complex flux dries out and it becomes very difficult, even impossible, to displace it. Moreover, a dry surrounding air (i.e. with a humidity below 40%) makes it hard, even impedes, the transferring of the isolated molecules and/or complexes toward a printing surface for subsequent analysis/detection. The later will be disclosed in more details afterwards.

In particular, the humidity is from 40 to 80% based on the maximal moisture content of the surrounding air. By “from 40 to 80%”, it is meant in the invention 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79% and 80%.

Particularly, the humidity is from 40 to 60% based on the maximal moisture content of the surrounding air, especially from 43 to 55% based on the maximal moisture content of the surrounding air.

Advantageously, the structured capture array is placed in a chamber, for example an hermetically sealed chamber.

Step b)

The complex fluid is then entirely covered by a covering means. The surface of the complex fluid located between the covering means and the capture array is not plane and is bent due to the surface tension of the complex fluid. Accordingly, the surface of the complex fluid on the capture array is higher than the surface of the complex fluid in contact with the covering means. The angle formed by the bending surface with the capture array is then closed and involved in the capture efficiency, as it will be described in more details below.

This bending surface is called meniscus. The complex fluid is surrounded by a meniscus which comprises a rear and a front, so-called rear meniscus and front meniscus. The front and the rear menisci are defined by the direction of the dragging step c). The front meniscus is located at the frontside when the complex fluid moves while the rear meniscus is located at the backside.

Step c)

In this step, the complex fluid is set in motion, advantageously at constant speed, by the dragging of the covering means or of the capture array. Importantly, the complex fluid stays hold between the covering means and the capture array all along step c) by means of the surface tension.

The movement of the complex fluid results in an efficient isolation of molecules and/or molecular complexes via hydrodynamical mechanisms. In more detail, the inventors unexpectedly identified that the displacement of the complex fluid at the surface of the topographically structured array results in the creation of a simple fluid, called the depletion zone, at the rear meniscus, similar in nature to the generation of a plasma for a blood sample. More specifically, the depletion zone is located at the junction between the rear meniscus, the structured capture array and the surrounding humid air, junction which is so-called the triple line. This depletion zone contains a high-concentration of low molecular weight molecules and/or molecular complexes with a radius of gyration less than 2 μm, including the molecules and/or molecular complexes of interest which have to be isolated.

The molecules and/or molecular complexes of interest are thus contained in a simple fluid (the depletion zone) and separated from the other bigger components of the complex fluid, i.e., the components with a radius of gyration higher than 2 μm. Accordingly, the other bigger components are not isolated in the structured capture array and remain in the complex fluid after step c). The whole components of the complex fluid are then at least partially isolated depending to their radius of gyration. The component having a radius of gyration below 2 μm but above the one of the molecules and/or molecular complexes of interest can also interestingly be isolated on the structured capture array thanks to the speed of dragging used during this step, as described in detailed below.

The isolation of the molecules and/or molecular complexes on the capture array becomes only possible because of the creation of such a simple fluid at the front meniscus. This is in particular due to the aforementioned variety of movements inside the fluid which drive the molecules and molecular complexes in all directions in a non-predictable manner.

On the contrary, in a simple fluid, a predictable and controlled flow drives the molecules and molecular complexes in a repeatable direction close to the front of the meniscus. In the invention, when this simple fluid at the triple line passes above the topographical features, the molecules and/or molecular complexes are pushed toward the bottom of the cavities thanks to the capillary forces until the front meniscus is passed over. Once the front meniscus is passed over, the molecules and/or molecular complexes remain trapped into the cavities with a small amount of the simple fluid. For molecules and molecular complexes in form of chains, such as polymers or DNA, one extremity of the chain can be trapped into one of the cavities while the rest of the molecule comprising the opposite extremity is elongated outside one of the cavities, on the plane surface, along the dragging direction.

In one embodiment of the invention, the molecules are biological molecules. In particular, the biological molecules are nucleic acid molecules. Particularly, the nucleic acid molecules are selected from the group comprising viral nucleic acid molecules, chromatin, circulating free DNA, RNA, linear DNA, linear RNA, circular DNA, circular RNA, single-stranded DNA, double-stranded DNA, G-quadruplex containing DNA, triple-strand DNA and tumoral DNA.

In one embodiment of the invention, the molecular complexes are biological complexes. In particular, the biological molecular complexes are selected in the group comprising vacuoles, lysosomes, transport vesicles, secretory vesicles, liposomes, ectosomes, microvesicles, virus, part of virus, exosomes and macro complex.

In one embodiment of the invention, the complex fluid is a biological fluid of an individual. In particular said biological fluid is selected in the group consisting of cerebrospinal fluid, pleural effusion, saliva, urine, blood, plasma and serum. Especially, the biological fluid is blood.

The process of the invention can be carried out with the raw complex fluid, i.e. a complex fluid which has not been submitted to any physical or chemical treatment or addition of one or more compounds (in solid, liquid or gas form).

Alternatively, before or at step a), the complex fluid is blended with a surfactant, in particular a non-ionic surfactant. Adding a non-ionic surfactant lowers the surface tension of the complex fluid. As a consequence, the angle formed by the meniscus of the complex fluid and the capture array is more closed than without the presence of a non-ionic surfactant. A more closed angle of the meniscus improves the capture efficiency because the molecules and/or molecular complexes in the depletion zone at the rear meniscus are closer to the cavities and better pushed into the cavities by the hydrodynamical flow.

The capture efficiency can be defined as the ratio between the number of cavities occupied by a molecular complex and the total number of cavities which have been uncovered by the rear meniscus.

Especially, before or at step a), the complex fluid is blended with 0.1 to 0.5% v/v Triton X100, particularly 0.3% v/v TritonX100. By “0.1 to 0.5% v/v” it is meant in the invention 0.10% v/v, 0.11% v/v, 0.12% v/v, 0.13% v/v, 0.14% v/v, 0.15% v/v, 0.16% v/v, 0.17% v/v, 0.18% v/v, 0.19% v/v, 0.20% v/v, 0.21% v/v, 0.22% v/v, 0.23% v/v, 0.24% v/v, 0.25% v/v, 0.26% v/v, 0.27% v/v, 0.28% v/v, 0.29% v/v, 0.30% v/v, 0.31% v/v, 0.32% v/v, 0.33% v/v, 0.34% v/v, 0.35% v/v, 0.36% v/v, 0.37% v/v, 0.38% v/v, 0.39% v/v, 0.40% v/v, 0.41% v/v, 0.42% v/v, 0.43% v/v, 0.44% v/v, 0.45% v/v, 0.46% v/v, 0.47% v/v, 0.48% v/v, 0.49% v/v and 0.50% v/v.

In one embodiment of the invention, before or at step a), the complex fluid is blended with at least one marking means configured for attaching the molecules and/or molecular complexes to be captured. The molecules and/or molecular complexes marked with the marking means can further be detected with the adequate detection method according to the used marking means. Marking means and adequate detection method are well-known by the skilled person.

In particular, the at least one marking means can be an antibody or a dye. The marking means can carry on a detection means or be recognised by a second marking means that carry on a detection means. The detection means is detected with the adequate detection method. The detection means can be a dye.

For example, when nucleic acid molecules have to be isolated, the marking means can be selected in the group comprising YOYO™−1 fluorescent dye of formula 1,1′-(4,4,8,8-tetramethyl-4,8-diazaundecamethylene)bis[4-[(3-methylbenzo-1,3-oxazol-2-yl)methylidene]-1,4-dihydroquinolinium] tetraiodide. Other compounds such as DAPI, Hoechst 33258, nucleic acid stains, genetic/epigenetic probes recognizing genetic sequences and epigenetic marks can also be used.

In case exosomes have to be isolated, the marking means can be Anti-CD63 antibodies or Anti-C81 antibodies, conjugated with a detection means being a fluorescein-based dye, or any other fluorescent dye.

The radius of gyration (Rg) of bare, i.e naked, DNA molecules in solution is easily estimated by a random coil model provided that these molecules contain sufficient nucleotides (number of base pairs (nbp) higher than 1000). By “naked DNA molecule”, it is meant in the invention a DNA molecule devoided of any protein, such as histones. The relationship is given by:

Rg(nm)=5.831\sqrt{nbp}

For lambda bacteriophage DNA, nbp equals 48502, and a radius of gyration of 1.28 μm is obtained.

For circulating DNA, consisting of small and long fragments, the radius of gyration ranges from 500 nm to 4 μm. Accordingly, the radius of gyration for proteins ranges from 1 nm to 20 nm. Tumoral microRNAs, which are small-size molecules, have radii of gyration close to those of small proteins (1 nm).

Chromosomal DNA fragments released by cells in the form of chromatin strands or nucleosome chains are rheologically more complex to describe because of the combination of DNA strands and the histones wrapped around them. Nevertheless, for small tumour fragments containing one or two histones (typical size of a histone octamer: 11 nm), the order of magnitude of the radius of gyration is about 10-20 nm. To give an idea of the range of variation in radii of gyration that can be expected, a chromatin strand of 1000 nbp has a radius of gyration around 100 nm, whereas a chromatin strand of 1 Mega nbp has a typical radius of gyration around 500 nm. Accordingly, for chromosomal fragments, the radius of gyration ranges from 10 nm to 1 μm.

For proteins in solution, the radius of gyration depends on the three-dimensional conformation of these proteins. For small proteins with a molecular weight around 10,000 Daltons, this radius of gyration is 1 nm. For larger proteins with a molecular weight of several million Daltons, the radius of gyration is around 20 nm.

Exosomes, when observed by electron microscopy further to their extraction, are spherical in shape. Their radius of gyration is therefore very close to the radius of this sphere. Data in current literature report radii of gyration between 15 and 150 nm.

The radius of gyration of the molecules and/or molecular complexes to be captured is the most relevant parameter in order to define the dimensions of the cavities of the capture array (width or diameter and depth), and in order to determine the dragging speed during step c).

In fact, a more efficient capture of a molecule and/or molecular complex on a topographical surface is obtained when the cavities are in the form of round wells and the radius of each cavity is comparable to the radius of gyration of the molecule and/or molecular complex. Indeed, the probability of capture on the topographical surface is maximum if their dimensions are equal. When the dimensions of the cavities are lower than those of the molecules and/or molecular complexes, they are too small for the molecule and/or molecular complex to get inside. When the dimensions of the cavities are higher than those of the molecules and/or molecular complexes, the probability of the molecule and/or molecular complex to be released from the cavities toward the depletion zone is increased, and more than one molecule and/or molecular complex can be trapped within one cavity which complicates a subsequent quantification/analysis. Nevertheless, using a combination of nanowells and microwells, as described below, improves the capture, and a fortiori in the microwells, of components of interest with a nanometric radius of gyration.

The cavities of the structured capture array may be nanowells and/or microwells.

As used herein, the term “microwell” and “nanowell” refers to a well-like structure of the structured capture array that has a depth or diameter (e.g., opening region at the surface) that is measured in micrometres or nanometres. For example, the microwells can have a diameter from 1 μm to 50 μm, and a depth from 1 μm to 50 μm. Regarding the nanowells, they can have a diameter from 10 nm to 900 nm and a depth from 10 nm to 900 nm.

By “from 1 μm to 50 μm”, it is meant in the invention 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm and 50 μm.

By “from 10 nm to 900 nm” it is meant in the invention 10 nm, 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm and 900 nm.

Regarding the dragging speed in step c), for a given assembly medium there is a specific capture time, T, required for the establishment of interactions between the molecules and/or molecular complexes and the topographical surface of the capture array allowing the molecules and/or molecular complexes to remain trapped within the cavities. The capture time turned out to depend on the nature of the complex fluid, the humidity and the affinity of the molecules and/or molecular complexes with the surface capture array.

The viscous phenomena that occur within the complex solution compete with the interactions between the molecule and/or the molecular complex to be captured and the capture array. Indeed, the molecules and/or molecular complexes need first to pass from the complex fluid to the simple fluid at the triple line. Consequently, the specific capture time is longer as the complex fluid becomes more “complex”, as defined above. In the same way, if the humidity is higher, the evaporation rate at the meniscus gets smaller and the transit of molecules and/or molecular complexes toward the triple line slows down, resulting in a higher capture time. Finally, the capture time also depends on the time required for stable interactions between the molecules and/or molecular complexes with the surface of the cavity to occur and thus depends on their affinity. Increasing the affinity by surface functionalization reduces the capture time. In the case of blood, for a humidity of 50% based on the maximal moisture and for surface cavities formed in pristine PolyDiMethylSiloxane, a typical value of τ is 50 ms.

The speed of dragging (ν) is defined by the following formula:

$$v ( μ m . {s}^{- 1} ) = \frac{{R}_{g} ( μ m )}{τ ( s )}$$

wherein R_{g} stands for radius of gyration. It is not necessary to know with high precision the radius of gyration of the molecule and/or molecular complex to be captured, an order of magnitude is sufficient for adjusting correctly the dragging speed. Optimal speed of dragging for a fluid containing a given molecule or molecular complex is different and lower in a drop of blood than in a simple fluid such as PBS buffer.

When the complex fluid is blood, the humidity at 50% based on the maximal moisture and the surface of the cavities formed in pristine PolyDiMethylSiloxane, the speed of dragging and the radius of gyration are related as follows:

V_{blood}(μm·s⁻¹)=20·R_{g}(μm)

From this relationship and the above-mentioned ranges of radius of gyration, the following speed of dragging in these particular conditions were determined:
Micro-RNA and proteins: from 0.4 μm/s to 20 μm/s
Exosomes: from 1 to 100 μm/s
Chromatin: from 1 to 50 μm/s
Circulating DNA: from 1 to 1000 μm/s

As seen here, category of molecules or molecular complexes can be defined by their ranges of radius of gyration. When the molecule and/or molecular complexes to be captured belongs to a different category, or have very different radius of gyration, it would be necessary to adapt the size of the cavities of the capture array, as described in more details below.

In one embodiment of the invention, the dragging at step c) is not carried out in a continuous manner but includes breaks of motion in order to improve the isolation of the molecules and/or molecular complexes. Especially, the breaks last for 1 second and are repeated every 30 seconds. The breaks give more time to the molecules and/or molecular complexes to pass from the depletion zone toward the cavities of the capture array. This is interesting especially when speed of dragging is low, i.e. below 10 μm·s⁻¹. Particularly, the breaks are performed when the rear meniscus is over cavities.

The process of the invention makes it possible isolating molecules and/or molecular complexes of different radius of gyration on the structured capture array. This can be carried out in one or two steps, as described here below.

In one embodiment of the invention, molecules and/or molecular complexes of different radius of gyration have to be isolated, and step c) is carried out at least two times at different speeds for each radius of gyration.

As seen above, the speed of isolation depends on the radius of gyration. Accordingly, if two molecules and/or molecular complexes which do not pertain to the same “category” have to be isolated, step c) is executed twice with the respective speed of isolation for each type of molecule and/or molecular complex to be isolated. This results in the successive isolation within the cavities of the structured capture array of the two molecules and/or molecular complexes.

Alternatively or in a complementary way, if molecules and/or molecular complexes of different radius of gyration have to be isolated, the structured capture array comprises at least a first and a second portions of topographical features wherein the first portion has larger cavities than the second one, such that step c) results in the spatial separation onto the structured capture array of the isolated molecules and/or molecular complexes of different radii of gyration. In particular the topographical features of the first portion are nanowells and the topographical features of the second portion are microwells.

This aspect of the invention is a combinatorial approach for the spatial isolation of different molecules and/or molecular complexes. Even if the different molecules and/or molecular complexes belong to the same “category” of isolation, which implies that the speed of isolation is the same, this aspect makes it possible to distinctively isolate them in space, thanks to the different size of cavities. In this case, step c) can be performed solely once. Understandably, step c) can be performed twice or more within this aspect, if needed, particularly if different speeds are needed.

Particularly, the structured capture array can present solely one first and one second portion. Especially, the said one second portion follows the said one first portion in the direction of the dragging. Accordingly, the molecules and/or molecular complex of lower radius of gyration are highly trapped into the first portion and rarely trapped into the second portion.

The structured capture array can also have several first and second portions. In particular, the structured capture array comprises an alternation of first and second portions in the direction of the dragging. Especially, the wells of the second portion can partially overlap the wells of the first portion. In particular, from 10 to 70% of the diameter of the wells of the second portion overlaps the diameter of the wells of the first portion. This configuration enables to capture at the same time and at the same location two types of molecules and/or molecular complexes, the one of large radius of gyration in the zone of the cavity of the first portion, the one of smaller radius of gyration in the zone of the second portion overlapping at the same cavity.

The structured capture array may comprise different regions with different distributions of first and second portions.

Particularly, the cavities of the first portion are microwells, and the cavities of the second portion are nanowells. The first portion may comprise different size of microwells, and the second portion may comprise different size of nanowells.

In particular, the cavities of the first portion and/or of the second portion are distributed through lines in a direction perpendicular to the dragging direction and/or in the dragging direction. Especially, the cavities of the first portion and/or of the second portion are evenly spaced in these both directions. Alternatively, the cavities of the first portion and/or of the second portion are more spaced in the dragging direction than in the perpendicular direction, especially for giving space to the chain molecules and/or molecular complexes to be elongated during step c).

In a particular embodiment, the cavities of the first portion are microwells, distributed through lines in the dragging direction and/or the perpendicular direction, have a diameter and a depth of 1 to 10 μm, especially 5 μm, and are spaced by 20 to 100 μm, especially 60 μm, in the dragging direction and by 5 to 15 μm, especially 9 μm, in the perpendicular direction.

Alternatively, or in a complementary way, the cavities of the second portion are nanowells, distributed through lines in the dragging direction and/or the direction perpendicular, have a diameter and a depth of 400 nm to 900 nm, especially 800 nm, and are spaced by 20 to 100 μm, especially 60 μm, in the dragging direction and by 200 to 600 nm, especially 450 nm, in the direction perpendicular. When combined with the above particular embodiment, the cavities of the first portions and those of the second portions are spaced by 10 to 50 μm, especially 30 μm, in the dragging direction.

In a particular embodiment, the cavities of the second portion are nanowells, distributed through lines in the dragging direction and the direction perpendicular, have a diameter and a depth of 500 nm and are spaced by 60 μm in the dragging direction and by 750 nm in the direction perpendicular.

In a particular embodiment, the cavities of the second portion are nanowells, distributed through lines in the dragging direction and the direction perpendicular, have a diameter and a depth of 800 nm and are spaced by 450 nm in both the dragging direction and the direction perpendicular.

In one embodiment of the invention, the topographical features have a hydrophobic surface. Especially, the hydrophobic surface comprises a polymer material. Preferably, said polymer material is selected from the group consisting of poly(dimethyl siloxane), parylene, poly(methylmethacrylate), polyethylenes, vinyls, and acrylates. This aspect of the invention is interesting in the case of the isolation of nucleic acid and is suitable for a solvent mediation of the isolated nucleic acid to a printing surface, as disclosed below.

In one embodiment of the invention, the material of the covering means is selected in the group comprising glass and oxidised silicon. In particular, the covering means is unfunctionalized. Alternatively, the covering means can be functionalized with bovine serum albumin or other non-sticking molecules.

The process of the invention can be coupled with immunological capture to locally modify the surface tension (local hydrophilicity) to enhance the molecule and/or complex molecule interaction with the structured capture array, their localization and attachment. to enhance the amount of captured molecule and/or molecular complexes.

In particular, the cavities of the structured capture array may be functionalised with a linking element configured for trapping the molecules and/or complexes. This linking element increases the efficiency of retaining the molecules and/or molecular complexes into the cavities. In particular, the linking element can be fixed to the bottom of the cavities of the structured capture array. Alternatively, the functionalisation of the said cavities can be carried out by performing steps a) to c) with a functionalisation solution comprising the said linking element, before performing steps a) to c) with the complex fluid comprising the molecules and/or complexes of interest. Accordingly, the linking element would be capture in the cavities before the molecules and/or complexes of interest be captured in the cavities. The different embodiments of the invention described in connection with the dragging of the complex fluid apply mutatis mutandis to the functionalisation solution. The dragging step with the functionalisation solution may be performed at 10 μm/s. The functionalisation solution may comprise Phosphate-buffered saline (PBS) with Triton-X, for example at a concentration of 0.5%.

Particularly, the linking element may be an antibody directed to the molecules and/or molecular complexes. Especially, when the molecular complexes to be isolated are exosomes the antibody is an antibody directed to the CD9, CD63 and/or CD81 epitope.

Once the molecules and/or molecular complexes are isolated within the cavities of the structured capture array, they can be directly detected on the capture array or after transfer on another support. Indeed, the process of the invention is compatible with all known characterization methods such as sequencing, fluorescence scanners or microscopy.

In particular, the process of the invention comprises a further step d):
d) contacting the surface of the structured capture array with a printing surface for transferring the trapped molecules and/or molecular complexes from the surface of the structured capture array to the printing surface.

In particular, the material of the printing surface is selected in the group comprising glass, silicon, supports for mass spectrometry, gold surfaces for quartz-crystal microbalance and gold surface for surface plasmon resonance.

In particular, the printing surface is functionalised with a transfer means configured to attach the isolated molecules and/or molecular complexes.

The transfer means may be an antibody directed to the isolated molecules and/or molecular complexes. For example, in case of isolated exosomes, the transfer means can be an antibody directed to the epitope CD9, CD63 and/or CD81.

The transfer means may be 3-aminopropyltriethoxysilane (APTES). APTES is commonly used to functionalize substrates because it can form an amine-reactive film that is tightly attached to the surface. APTES transfer means is especially used in case of transfer of nucleic acids. The substrates can be in particular plasma-activated glass (hydroxyle groups) or APTMS (tri-methoxysilane).

The transfer means may be (3-glycidoxypropyl)trimethoxysilane (GPTMS). GPTMS transfer means is especially used in case of transfer of liposomes.

When the captured molecules and/or complex molecules are dried in the cavities or when the printing surface is hydrophobic, the printing surface can be covered with a thin film of solvent which by evaporation, will accelerate the transfer of the molecules and/or complex molecules. The solvent can be ethanol, deionized water or saline buffer or a mixture of the two.

The invention also relates to the use of a structured capture array for in vitro isolating molecules and/or molecular complexes having a radius of gyration smaller than 2 μm from a complex fluid comprising numerous components, wherein the structured capture array having topographical features in the form of a plurality of plane surfaces in-between cavities.

### LEGEND TO THE FIGURES

FIG. 1 is a set of three epifluorescence images (A to C) of DNA strands isolated from a blood sample on a structured capture array and printed on a functionalised coverslip. Each white line represents one or an assembly of DNA strands. In FIG. 1A, the speed of dragging was 1 mm·s⁻¹. In FIG. 1B, the speed of dragging was 200 μm·s⁻¹. In FIG. 1C, the speed of dragging was 20 μm·s⁻¹.

FIG. 2 is a set of two epifluorescence images (A and B) of circulating DNA strands isolated from a blood sample on a structured capture array and printed on a functionalised coverslip. Each white line represents one or an assembly of DNA strands.

FIG. 3 is an epifluorescence image of liposomes isolated from a blood sample on a structured capture array and printed on a functionalised coverslip. Each solid circle represents a plurality of liposomes and fits the dimension of the round well where the liposomes were isolated.

FIG. 4 represents two stamps' configurations and the respective capture of DNA strands and fluorescent nanoparticles. FIG. 4a) represents a “non-sequential” configuration of a stamp observed with a Scanning Electron Microscopes (SEM) and FIG. 4c) is an epifluorescence image of DNA strands and fluorescent nanoparticles isolated from a blood sample on the said stamp. White circles surround the captured fluorescent nanoparticles. FIG. 4b) represents a “non-sequential” configuration of a stamp observed with a Scanning Electron Microscopes (SEM) and FIG. 4d) is an epifluorescence image of DNA strands and fluorescent nanoparticles isolated from a blood sample on the said stamp. White circles surround the captured fluorescent nanoparticles.

FIG. 5 represents assemblies of fluorescent polystyrene (PS) nanoparticles at different speeds. FIG. 5a) is an epifluorescence image of polystyrene nanoparticles captured on a non-sequential stamp at 2 μm/s. FIG. 5b) is an epifluorescence image of polystyrene nanoparticles captured on a non-sequential stamp at 3 μm/s. FIG. 5c) is an epifluorescence image of polystyrene nanoparticles captured on a non-sequential stamp at 5 μm/s. FIG. 5d) is an epifluorescence image of polystyrene nanoparticles captured on a non-sequential stamp at 7 μm/s. FIG. 5e) is an histogram representing the average number of isolated nanoparticles aggregates within 20 micro cavities as function of the assembly speed. FIG. 5f) is a histogram representing the average number of isolated nanoparticles aggregates within 10650 nanocavities as function of the assembly speed.

FIG. 6 relates to comparison of fibers of polymerized plasma proteins and DNA strands observed in fluorescence and in bright field. FIG. 6a) is an epifluorescence image of assembled fibers from unspiked blood (without spiked DNA strands) captured on a non-sequential stamp at 10 μm/s. FIG. 6b) is a bright field image of the assembled fibers observed at FIG. 6a). FIG. 6c) is an epifluorescence image of assembled DNA strands from spiked blood captured on a non-sequential stamp at 10 μm/s. FIG. 6d) is a bright field image of the assembled DNA strands observed at FIG. 6c).

FIG. 7 relates to an analysis of the influence of the Triton concentration on the occupation rate of polymerized plasma proteins fibers. FIG. 7a) is an epifluorescence image of assembled fibers from unspiked blood (without spiked DNA strands) with 0.5% Triton-X captured on a non-sequential stamp at 2 μm/s. FIG. 7b) is an epifluorescence image of assembled fibers from unspiked blood (without spiked DNA strands) with 0,25% Triton-X captured on a non-sequential stamp at 2 μm/s. FIG. 7c) is an epifluorescence image of assembled fibers from unspiked blood (without spiked DNA strands) with 0,125% Triton-X captured on a non-sequential stamp at 2 μm/s. FIG. 7d) is a histogram representing the occupation rate of the cavities of the stamp by the fibers as function of the Triton-X concentration (TX).

FIG. 8 relates to Biofunctionalization of the bottom of surface cavities through capillary assembly. FIG. 8a) is an epifluorescence image of a control experiment wherein an assembly of a PBS solution with 0.5% of Triton-X at 10 μm/s on a non-sequential stamp. FIG. 8b) is epifluorescence image of an assembly of a PBS solution with 0.5% of Triton-X and a fluorescent labelled anti-CD81 antibody at 20 μg/mL at 10 μm/s on a non-sequential stamp.

FIG. 9 relates to assembly of exosomes from whole blood. FIG. 9a) represents a 5 μm cavity from a non-sequential stamp observed by SEM after assembling a control solution. FIG. 9b) represents a 5 μm cavity from a non-sequential stamp observed by SEM after assembling of exosomes from spiked blood on a non-sequential stamp. FIG. 9c) is an epifluorescence image of the stamp after assembling the control solution. No fluorescence is observed on the stamp. FIG. 9d) is an epifluorescence image of the stamp after assembling the exosomes from spiked blood and incubated with a florescent antibody direct to the exosomes. Exosomes are revealed (white spots) on the cavities of the stamp.

FIG. 10 relates to Different methods for characterizing the combined capture of exosomes and circulating free DNA (cfDNA). FIGS. 10a), 10b and 10c are images obtained by SEM at different magnifications, after assembly of sample 1 on a non-sequential stamp at 10 μm/s. Exosomes are observable as white dots inside the cavities. FIG. 10d) is an epifluorescence image of DNA strands and exosomes after assembling of sample 2. Captured exosomes are highlighted by white circles. FIG. 10e) is an epifluorescence image of captured DNA strands after assembling of sample 3. FIG. 10f) is an epifluorescence image of captured exosomes (highlighted by white circles) after assembling of sample 3.

### EXAMPLES

### Example 1: Isolation of DNA Strands from a Blood Sample Enriched in DNA Extract

1. Preparation of a Polydimethylsiloxane (PDMS) Stamp

The structured capture array is a PDMS stamp. The topographical features of the PDMS stamp are formed using a silicon mould. The silicon mould comprises several pillars with a 20 μm space between them in order to form wells in the PDMS stamp. The PDMS is prepared by using Sylgard™ 184 Kit. In accordance with the notes on completion, 1 dose of curing reagent is mixed with 10 doses of base. The curing agent contains Dimethyl siloxane, dimethylivinyl terminated (CAS Number: 68083-19-2), Dimethylvinylated and trimethylated silica (CAS Number: 68988-89-6), Tetra (trimethoxysiloxy) silane (CAS Number: 3555-47-3) and Ethyl benzene (CAS Number: 100-41-4). The base contains Dimethyl, methylhydrogen siloxane (CAS Number: 68037-59-2), Dimethyl siloxane, dimethylvinyl terminated (CAS Number: 68083-19-2), Dimethylvinylated and trimethylated silica (CAS Number: 68988-89-6), Tetramethyl tetravinyl cyclotetra siloxane (CAS Number: 2554-06-5, Ethyl benzene (CAS Number: 100-41-4). This PDMS mixture is then poured onto the mould, baked at 80° C. during 2 hours to polymerize and become solid.

2. Preparation of a Functionalized Coverslip for Printing

a. Cleaning of the Coverslip

First, one side of a glass coverslip is cleaned up with a solution of acetone, then with a solution of ethanol and finally with a solution of deionised water. Then, the washed coverslip side is placed up on a paper towel and dried with nitrogen by means of an air gun. Thereafter, the coverslip is moved to a new spot on the towel and dried again with the gun. Afterwards, the coverslip is taken with tweezers and nitrogen is blown from the side of the coverslip to get rid of any water residual. Finally, a radio frequency plasma treatment is performed during 5 min at 0.6 mbar air and 100% power (50 W).

b. Functionalization of the Coverslip with an APTES Solution (1% Silane in 95% EtOH/5% ddH₂O)

A hot plate is preheated to 140° C. A solvent is prepared by mixing 47.5 ml of ethanol and 2.5 ml of distilled water. 0.5 mL of APTES solution is taken using a syringe by inverting it and accounting for the volume of the air bubble forming inside the syringe. The solvent and the APTES solution are mixed in the glass dish and then covered with aluminium foil to limit evaporation for 5 minutes. The air atmosphere within the dish is replaced by nitrogen to limit air contact during hydrolysis. The aluminium foil is removed to allow the introduction of the plasma activated coverslip inside the dish for 20 minutes. The functionalised coverslip is then removed, cleaned thoroughly with ethanol and ddH₂O, dried with a nitrogen gun and finally put on a hot plate at 140° C. for 5 minutes.

3. Preparation of Triton-X100 and YOYO-1 Solutions

A 10% Triton-X100 solution is prepared by mixing 1 ml of Triton-X with 9 ml of PBS in a 15 mL Falcon tube.

YOYO-1 is a nucleic acid stain. A 1:10 dilution of the stock YOYO-1 solution is prepared by diluting with PBS a 1 mM stock solution.

4. Preparation of the DNA Extract

Lambda phage DNA extracts were acquired from New England Biolabs (NEB)

5. Preparation of the Assembly Solution

Blood was obtained from the Etablissement Français du Sang (EFS), collected in EDTA coated tubes to prevent clotting.

The assembly solution was prepared by mixing 45.25 μl of blood with 2.5 μl of the DNA extract, 0.75 μl of the solution of YOYO-1 and 1.5 μl of the solution of Triton-X100. The final concentration in the assembly solution are the following:
Triton-X100: 0.3% v/v,
YOYO-1: 7.5 μM,
DNA extract: 25 μg/ml.

6. Mounting the Assembly Solution on the PDMS Stamp

The temperature is set at 20° C. and the humidity at 40% as measured with a numerical hygrometer. A coverslip is cleaned as described at point 2.a. With the topographical features facing up, the PDMS stamps is placed on a PDMS Petri dish with its long side perpendicular to the movement of dragging (long side horizontal). The cleaned coverslip is placed and held at approximately 2-3 mm above the PDMS stamp. 40 μL of the assembly solution is placed between the PDMS stamp and the coverslip. Then the droplet is spread evenly all along the topographical features of one the short side of the PDMS stamp.

7. Isolation and Printing of the Extract DNA

The coverslip is moved in the direction of the long side of the PDMS stamp at speeds of 20 μm/s, 200 μm/s and 1 mm/s. Once the other short side of the stamp is reached, the stamp is removed and the remaining droplet is wicked away using a paper. All of the water residuals are removed to avoid any spread upon contact with the functionalised coverslip. The topographical side of the PDMS stamp is then placed into contact with the functionalised side of the functionalised coverslip for 1 min. Thereafter, the PDMS stamp is removed and stored in dark conditions.

8. Epifluorescence Microscopy

Samples are observed at x100 magnification using an inverted microscope (Olympus, exposure: 30 ms; camera gain: 100; cyan light; laser power 30; Zeiss, Camera gain: 3; exposure time: 200 ms; laser power: 100%; cyan light).

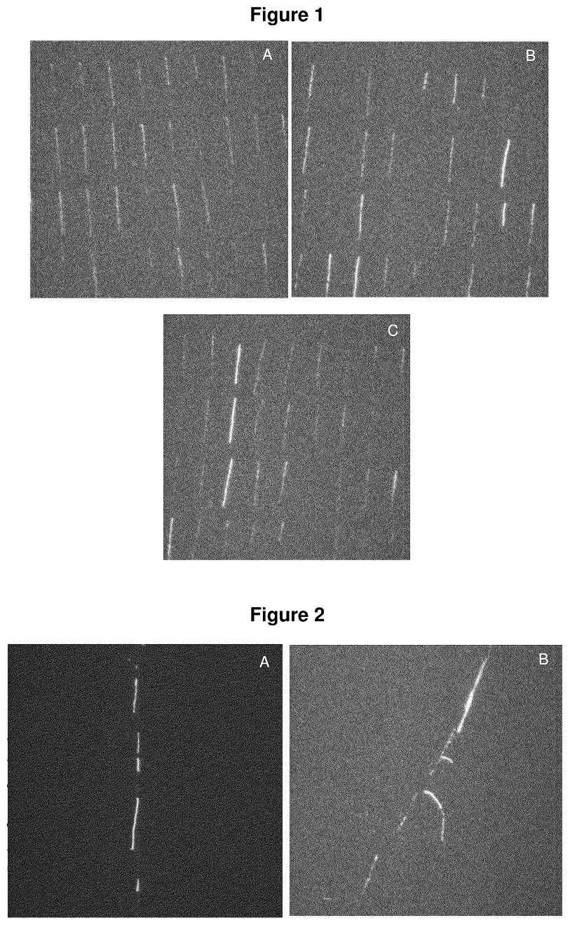

The results obtained for each speed are represented at FIGS. 1A to 1C. The white lines correspond to the elongated DNA strands printed at the surface of the functionalised coverslip. On the pictures, one can see some DNA strands that seem thicker and brighter than the others. The brighter strands correspond to a set of strands and the lighter strands to single strands, which would explain the brightness and thickness.

The inventors obtained a better reproducibility and a better coverage of the functionalised coverslip (resulting from a better isolation of the DNA strands into the wells of the PDMS stamp) at a 20 μm/s speed of dragging.

### Example 2: Isolation of Circulating DNA from a Blood Sample

The aim of this example is to isolate circulating DNA from a blood sample pertaining to a patient afflicted by cancer.

Points 1. to 3. and 5. to 8. of Example 1 were reproduced, except that
in point 5, no DNA was added to the assembly solution and that 47 μl of a blood sample recovered from a clinical trial with patients afflicted by cancer is mixed with 1.5 μl of the solution of YOYO-1 and 1.5 μl of the solution of Triton-X100, and that
in point 7, the speed at step a) is 20 μm/s.

The results are represented on FIGS. 2A and 2B.

### Example 3: Isolation of liposomes added to a blood sample

Points 1. to 3., 6. and 7. of Example 1 were reproduced, except that
in point 2., the cleaned coverslip is functionalized with GPTMS according to the below protocol;
in point 7, the speed at step a) is 20 μm/s.

Points 4. and 5. of Example 1 are replaced by the below points 2. and 3. respectively.

1. Functionalisation of the Coverslip with GPTMS

1.25 ml of a solution of GPTMS is mixed with 48.75 ml of pure ethanol. The plasma activated coverslip's side is laid on the mixture for 30 min. The functionalised coverslip's side is cleaned thoroughly with ethanol only and dry slides with an air gun.

2. Preparation of Liposomes

The lipids used for this protocol are phosphatidylcholines (POPC) and 1-palmitoyl-2-{6-[(7-nitro-2-1,3-benzoxadiazol-4-yl)amino]hexanoyl}-sn-glycero-3-phosphocholine (NBD-PC) from Avanti Polar Lipids. NBD-PC is a fluorescent lipid used to see the liposomes in epifluorescence (excitation wavelength 460 nm; emission wavelength 534 nm).

a. Preparation of Lipids Solutions

The phospholipids are stored at −20° C. in solution in chloroform (Sigma Aldrich), at a concentration of 10 mg/ml for POPC and 1 mg/ml for NBD-PC.

b. Preparation of Phospholipids

100 μl of POPC solution is mixed with 10 μl of NBD-PC solution in a 4 ml glass flask. Then the flask is heated at 55° C. in a dry bath while an air flow is creating using an air gun pointing inside the flask in order to make the chloroform evaporate. The flask is thereafter placed in a vacuum chamber for 2 hours to make sure all the chloroform is evaporated. The chamber is covered with aluminium foil to avoid fluorophore destruction by daylight. Finally, the flask is sealed with a parafilm and stored at 4° C. if not used right away.

c. Suspension of Lipids to Obtain Multilamellar Vesicles

Add 0.5 ml of PBS is added in the flask and vortexed until complete homogenisation. Formation of 80 nm diameter small unilamellar vesicles (SUV) is performed by extrusion using Avanti Polar Lipids “mini extruder kit”.

The SUV suspension is at 2 mg/ml concentration and can be stored at 4° C. and used within 3 days.

3. Preparation of the Assembly Solution

The assembly solution was prepared by mixing 25 μl of blood with 22.5 μl of the liposome solution and 2.5 μl of the solution of Triton-X100.

4. Epifluorescence Microscopy

Samples are observed at x100 magnification using an inverted microscope (Olympus, exposure: 30 ms; camera gain: 100; cyan light; laser power 30; Zeiss, Camera gain: 3; exposure time: 200 ms; laser power: 100%; cyan light).

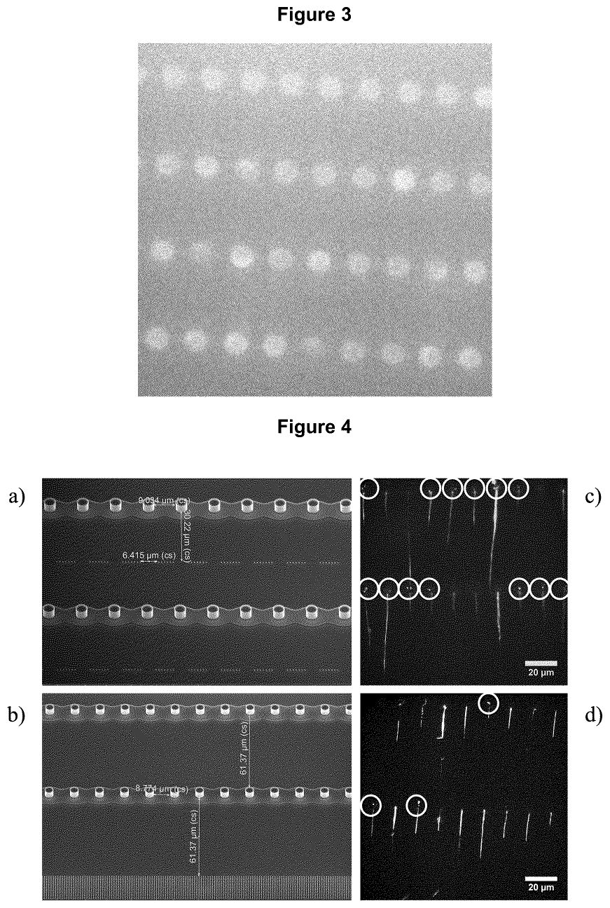

The results for a speed of 10 μm/s are represented in FIG. 3. This figure shows that the liposomes were efficiently isolated into the wells and transferred onto the functionalised coverslip.

### Example 4: Configurations of Stamp Patterns

1. Objectives of the Experiment

New stamps have been built in order to implement a combinatorial liquid biopsy capture. The stamp used in Examples 1 to 3 contained only micro cavities with diameter of 5 μm. The objective of this example was to develop new configurations of stamps which contains both micro cavities of 5 μm, adapted for the DNA strands capture, and nano cavities of 500 nm, adapted for nanoparticles capture, in particular exosome.

2. Materials and Methods

Two configurations of stamps patterns were tested. The first one is called “non-sequential” and consists in alternating micro patterns with nano patterns along the dragging direction. The second one, “sequential”, is split in two: one half of the stamp surface is equipped with micro patterns and the second half with nano patterns. Both configurations are visible in FIGS. 4a) and 4b).

Thanks to microfabrication's processes, new moulds composed with both configurations were fabricated in a clean room. Each mould consisted in a 4 inches wafer of silicon and on this wafer, different chips were defined. Half of the chips corresponded to non-sequential configuration and the other half to sequential configuration. PDMS (Polydimethylsiloxane) replica were produce by conventional molding process, after cross-linking the stamps were cut according to chip borders. After unmolding, the different stamps were composed of micro cavities and nano cavities either in sequential or in non-sequential configurations.

In order to investigate the suitability of these stamps for combinatorial assembly, a solution of blood spiked with 100 nm fluorescent polystyrene nanoparticles (concentration of 2,5 μg/mL=5×10⁹ particles/mL) and DNA strands (25 μg/mL) with 0.5% of Triton-X and 0,75 μM of YOYO-1 was prepared. A drop of 35 μL of this solution was assembled at 10 μm/s then at 2 μm/s on each configuration.

Captured nanoparticles and DNA strands are observed at x100 magnification using an inverted microscope (Zeiss, Camera gain: 3; exposure time: 200 ms; laser power: 100%; cyan light).

Stamps configurations are observed with a Scanning Electron Microscopes (SEM Helios 600i FEI, Acceleration Voltage 15 kV, e-beam current 86 pA, secondary electron signal).

3. Results

The results are presented on FIGS. 4c) and 4d). In both configurations, DNA strands and nanoparticles are assembled. The nanoparticles are observable in micro cavities in both configurations (they are indicated by the circles). Inside nano-cavities the nanoparticles are not detected by fluorescence. The results obtained between the two configurations are equivalent: the DNA occupation rate, the length and the good state of the captured DNA strands, as well as the ratio of occupied cavity with nanoparticles.

### Example 5: Nanoparticles Assembly

1. Objectives of the Experiment

Exosomes are extracellular vesicles exhibiting typical sizes between 30 and 140 nm. They contain proteins, miRNA, DNA and other biomarkers bringing information about the disease. They are considered as a novel biomarker and their study in a cancer research context is more and more developed. However, due to their small size, they are difficult to isolate. In order to determine the optimal parameters for their capture, exosomes were micmicked with 100 nm fluorescent polystyrene nanoparticles in this experiment. The objective of this experiment is thus to implement the assembly of nanoparticles in blood at a concentration closed to realistic exosomes concentration and to determine the optimum speed of assembly.

2. Materials and Methods

A solution of blood spiked with 100 nm fluorescent polystyrene nanoparticles (2,5 μg/mL=10⁹ particles/mL) with 0.5% of Triton-X is prepared. A drop of 35 μL of this solution was assembled at 2, 3, 5 and 7 μm/s on sequential and non-sequential stamps as described in example 4, in order to determine the optimal assembly speed. After assembly, each stamp was put on a clean coverslip and observed on the microscope through this coverslip.

Captured nanoparticles are observed at x100 magnification using an inverted microscope (Zeiss, Camera gain: 3; exposure time: 200 ms; laser power: 100%; cyan light).

3. Results

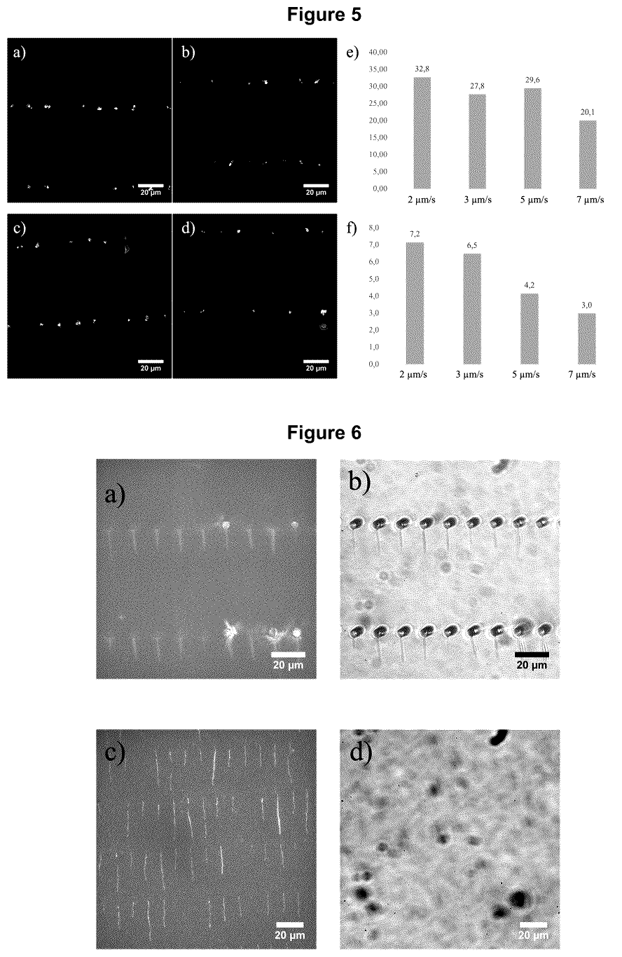

The results are shown in FIG. 5. Nanoparticles were isolated in micro cavities as well as in nano cavities. The graphs 5e) and 5f) show a decrease of the number of isolated nanoparticles aggregates in both micro and nano cavities with the increasing of the speed, which enables to establish a link between scanning speed and quantity of captured nanoparticles.

4. Conclusion

Nano species such as nanoparticles were assembled from whole blood in all type of cavities (micro and nano). Optimizing the speed of dragging optimizes the quantity of isolated nanoparticles. Here, the optimum speed was given at 2 μm/s, despite that satisfying result can be obtained at higher speeds.

### Example 6: Capture of Plasma Proteins Fibers

1. Objectives of the Experiment

At multiple occasions, some fibers were assembled during experiments in whole blood without any spiking of DNA molecules. They are different from DNA because through SEM and optical fluorescence observation they appear to be thicker, more diffuse and less fluorescent. The inventors hypothesized these fibers were formed during capture by capillary assisted polymerization of plasma proteins such as fibrine. The objectives of the following experiments are to confirm these fibers are not DNA molecules and to determine a way to limit them, if desired.

2. Materials and Methods

A solution of blood with 0.5% Triton-X is prepared and an assembly on a non-sequential stamp, as described in example 4, at 10 μm/s with a drop of 35 μL of this solution is carried out. At the end of the assembly, the stamp is observed through a glass coverslip on the microscope described in example 4. The result is compared to another sample (at 10 μm/s with a drop of 35 μl) corresponding to the assembly in blood spiked with lambda-phage DNA strands dyed with YOYO-1 at 0,75 μM. Both samples are visualized in fluorescence and in bright field on the microscope described in example 4. As DNA strands cannot be observed in bright field under these conditions while protein fibers do because of their thicker structure which increases light diffusion, that makes it possible to distinguish DNA strands from protein fibers. In order to determine a way to limit the apparition of these protein fibers, three solutions of blood with 0.5%, 0,25% or 0,125% Triton-X were prepared and assemblies at 2 μm/s are performed on a non-sequential stamp, as described in example 4. The three stamps (one per concentration) are observed on a microscope through a coverslip and compared.

3. Results

FIG. 6a) represents the assembled fibers from unspiked blood in fluorescence and FIG. 1b) in bright field. One can see that fibers of polymerized plasma proteins are observable at the same locations in both images. It means that these captured objects are visible in bright field.

FIGS. 6c) and 6d) represent the assembled spiked DNA strands dyed with YOYO-1 from blood in fluorescence (6c)) and in bright field (6d)). In FIG. 6d), no strand appeared, confirming DNA strands are only observable in fluorescence.

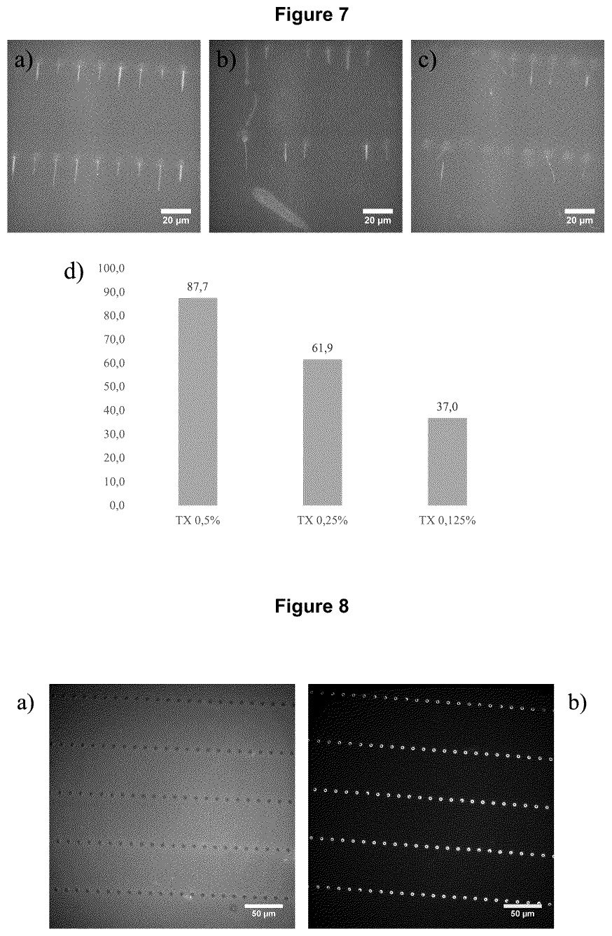

FIG. 7 represents assemblies of unspiked blood at different Triton-X concentrations. On FIGS. 7a), 7b) and 7c), which are respectively assemblies at 0.5%, 0,25% and 0,125% of Triton-X in blood, one can see that the number of observed fibers decreases with the decrease of Triton-X concentration. The graph on FIG. 7d) confirms this result since the occupation rate decreases when the Triton-X concentrations decreases.

Other experiments (not described here) proved that the speed also has an influence on the quantity of fibers. Indeed, there are more fibers of polymerized plasma proteins at 2 μm/s than at 10 μm/s.

4. Conclusion

It can be affirmed that the fibers assembled from blood are not DNA strands, since the latter cannot be observed in bright field.

A link between the occupation rate of polymerized plasma proteins fibers and Triton-X concentration can be established, since the number of assembled fibers decreases if the Triton-X concentration decreases. The speed is also an important factor for the assembly of those fibers since their number decreases when the speed increases.

In conclusion, very low scanning velocity combined with high Triton-X concentration favors the formation of protein fibers in whole blood. If such phenomenon needs to be limited, the operation parameters need to be tuned such as speed is set at 10 μm/s or more and Triton-X concentration is used at a concentration below 0,125%.

### Example 7: Biofunctionalization of the Bottom of the Surface Cavities with a Specific Antibody

1. Objectives of the Experiment

The objective of this experiment was to functionalize the bottom of the surface cavities in order to facilitate the exosomes assembly inside the cavities while preventing their adsorption on the top surface of the stamp. This functionalization has been already attempted by incubation of an antibody on the stamp for one hour. A printing of the stamp was then realized by putting the stamp in contact with different coverslips, one after another, in order to progressively remove the antibody molecules adsorbed on the top surface. However, the results were not excellent and reproducible from stamp to stamp. The inventors thus decided to functionalize the bottom cavities by capillary assembly. In other words, they used the same principle of biomarker capture but instead of manipulating a blood microvolume, they manipulate a microvolume of a solution containing the selected antibody. Capillary effects thus drive these molecules inside the cavities and not on the top surface of the stamp. The described experiment demonstrates this principle.

2. Materials and Methods

A solution of PBS (1X) is prepared with 0.5% of Triton-X and with a fluorescent labelled anti-CD81 antibody at 20 μg/mL in PBS. CD81 is a protein inserted inside the envelope of exosomes. A drop of 35 μL of this solution is then assembled on the surface of a non-sequential stamp, as described in Example 4, at a speed of 10 μm/s.

A control solution with only PBS and 0.5% of Triton-X was also prepared. An assembly is realized at 10 μm/s on another non-sequential stamp.

Both stamps, the control one and the one with the antibody are observed with the optical microscope described in Example 4 through a coverslip and compared.

3. Results

The results are presented on FIG. 8. On the left picture (control), no fluorescence is observed. On the contrary, fluorescents dots are visible on the right picture sample attesting for the immobilization of the anti-body at the bottom of each micro-cavities present on the stamp. Interestingly, one can note that the fluorescence is only visible inside the cavities, not on the top surface.

4. Conclusion

Thanks to the capillary assembly technique, the antibody can be placed only in the bottom of the cavities. Advantageously, the repartition of the antibody is homogeneous in all the cavities and solely one step is to be performed to functionalize the cavities. Moreover, this functionalization does not require cleaning step(s) since the antibody is only present inside the cavities.

### Example 8: Exosome Capture from Whole Blood

1. Objectives of the Experiment

The objective of this experiment is to capture exosomes from whole blood by capillary assembly on a bio-functionalized stamp (bottom cavities). The inventors characterized the capture by optical fluorescence microscopy and by Scanning Electron Microscopy (SEM).

2. Materials and Methods

Three solutions were prepared:
a functionalization solution comprising PBS with 0.5% of Triton-X and anti-CD81 antibody at 20 μg/mL in PBS,
an exosomes solution comprising a blood sample enriched with exosomes at a concentration close to 10⁹/mL and 0,5%, of Triton-X and
a control solution which is composed of unspiked blood and 0.5% of Triton-X.

For both optical fluorescence and SEM observation, two samples were characterized:
1) A control (unspiked blood)
2) An assembly of exosomes from spiked blood on a biofunctionalized stamp (bottom cavities)

For the control, a drop of 35 μL of the anti-CD81 solution is assembled on a non-sequential stamp at 10 μm/s. Immediately after the functionalization, another assembly is performed at 2 μm/s with a drop of 35 μL of the control solution.

For the exosomes assembly on a biofunctionalized stamp, a drop of 35 μL of the anti-CD81 solution is assembled on a non-sequential stamp at 10 μm/s. Immediately after the functionalization, another assembly is performed at 2 μm/s with a drop of 35 μL of the exosomes solution.

When the resulting capture is observed in fluorescence, an incubation is carried out for one hour with a solution of fluorescein isothiocyanate (FITC) anti-CD63 (Thermofisher, MA1-19602) diluted at 1:100 in volume in PBS at the end of the assembly of the exosomes solution or the control solution. This secondary fluorescent antibody reveals the presence of exosomes, the CD63 being a protein commonly found in the envelope of exosomes. After the incubation, the stamp is rinsed four times with PBS.

SEM observations were performed directly without performing any incubation with the fluorescent secondary antibody (SEM Helios 600i FEI, Acceleration Voltage 15 kV, e-beam current 86 pA, secondary electron signal).

3. Results

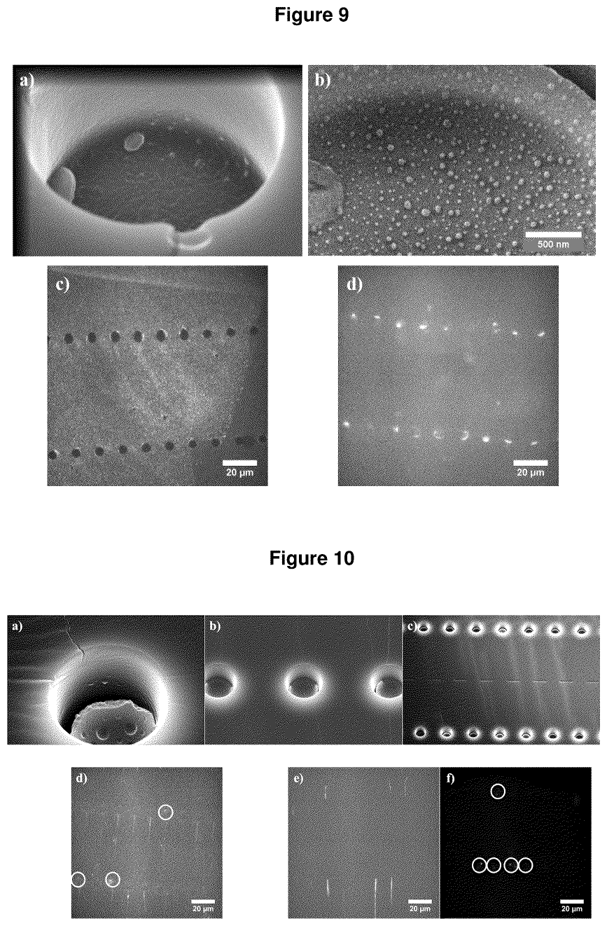

The results are presented on FIG. 9. On FIG. 9a) (the control), by SEM no element compatible with exosome shape and dimension can be observed at the bottom of the cavities. Accordingly, no exosome was captured. This is confirmed by FIG. 9c) where the control is observed in fluorescence after incubation and no signal coming from the bottom of the cavities can be observed. The background fluorescence is only at the surface of the stamp.

On FIG. 9b) (exosomes on functionalized stamp), nano-spheres are clearly observed at the bottom of the cavities. They correspond to exosomes according to their size and typical contrast. Indeed, exosomes are vesicles with typical sizes between 30 and 140 nm and they are recognizable in electron microscopy thanks to a donut like contrast in their center. This is also confirmed by FIG. 9d) where fluorescence dots are located at the bottom of the cavities, when the focus plane is adjusted at that location, revealing the presence of exosomes inside the cavities.

4. Conclusion

By comparing the different images, obtained by fluorescence or SEM, it can be affirmed that, by functionalizing the bottom of the cavities, exosomes can be assembled. The control samples demonstrates that nothing is observed inside the cavities if exosomes are not spiked into blood.

### Example 9: Different Methods for Combining the Capture of Exosomes and Circulating Free DNA (cfDNA)

1. Objectives of the Experiment:

The objective of this experiment is now to perform the combinatory capture with DNA strands and exosomes both spiked inside whole blood. Captured samples were observed in fluorescence and Scanning Electron Microscopy. However, in order to observe exosomes in fluorescence, they need to be dyed with a secondary fluorescent antibody by incubation. This incubation is problematic for the already assembled DNA strands since they can be resuspended in the solution during secondary antibody incubation. Here the inventors solved this problem with the following experiments:
a combinatory capture in one step (without incubation of a secondary labelling antibody for exosomes),
a combinatory capture using combination of both biomarkers in fluorescence (with the incubation of the secondary labelling antibody for exosomes)

2. Materials and Methods:

A solution of PBS with 0.5% of Triton-X and an anti-CD81 antibody at 20 μg/mL in PBS was prepared. Another solution of blood with DNA strands at 25 μg/mL and exosomes (approximate concentration of 10⁹ mL⁻¹) with 0.5% Triton-X and 0,75 OA of YOYO-1 was prepared.

Three samples were realized:
1. one for SEM observation,
2. one with both biomarkers (DNA strands and exosomes) assembled on a same stamp in order to be observed together by fluorescence microscopy,
3. one with both biomarkers first assembled on a same stamp but the DNA strands are transferred on an APTES functionalized coverslip, as previously described in Example 1, before the exosomes incubation with a fluorescent antibody. Hence, the DNA strands are on the printed coverslip while the exosomes still remain inside the cavities of the stamp. The two biomarkers are finally observed separately by fluorescence microscopy.

For sample 1, an assembly of the bio-functionalization solution with the anti-CD81 antibody is performed at 10 μm/s on a non-sequential stamp as described in Example 4. Another assembly is then realized with a drop of 35 μL of the spiked blood with exosomes and DNA strands at 10 μm/s. After the assemblies, a thin metallic film of 5 nm of Au/Pd is deposited at the stamp surface. The sample is then observed by SEM (SEM Hélios 600i FEI, Acceleration Voltage 15 kV, e-beam current 86 pA, secondary electron signal).

For sample 2, an assembly of the functionalization solution with the anti-CD81 antibody is performed at 10 μm/s on a non-sequential stamp as described in Example 4. Another assembly is then realized with a drop of 35 μL of the spiked blood with exosomes and DNA strands at 2 μm/s. An incubation is carried out for one hour with a solution of a secondary labelled FITC anti-CD63 (Thermofisher, MA1-19602) diluted at 1:100 in volume in PBS. After the incubation, the stamp is rinsed four times with PBS. Another assembly is then performed with a drop of 35 μL of the same spiked blood with exosomes and DNA strands at 10 μm/s, in order to capture fresh DNA strands and compensate those re-suspended during the incubation of the secondary antibody.

For sample 3, an assembly of the functionalization solution with the anti-CD81 antibody was performed at 10 μm/s on a non-sequential stamp as described in Example 4. Another assembly is then realized with a drop of 35 μL of spiked blood with exosomes and DNA strands at 2 μm/s. The stamp is pressed on a APTES (3-Aminopropyl-triethoxysilane) functionalized coverslip as described in Example 1 for one minute in order to transfer the assembled DNA strands by electrostatic interactions. After the transfer, an incubation is carried out on the stamp for one hour with a solution of a secondary labelled FITC anti-CD63 (Thermofisher, MA1-19602) diluted at 1:100 in volume in PBS in order to label the exosomes captured inside the cavities. After the incubation, the stamp is rinsed four times with PBS. DNA strands were observed, with the microscope described on example 4, the coverslip after their transfer by nano-contact printing while the exosomes were observed directly on the stamp with the said microscope through a protecting coverslip.

Results

SEM observations of sample 1 are presented on FIGS. 10a), 10b) and 10c). As one can see, exosomes were assembled at the bottom of the cavities (white dots). DNA strands were also observable as long white line starting from the cavities on the deposited thin metallic film used in SEM observation.

The fluorescence characterization of both biomarkers on the same support (sample 2) is represented on FIG. 10d). As one can see, exosomes and DNA strands were well characterized by fluorescence on the same image.

The biomarkers can also be observed separately (sample 3), as shown on FIGS. 10e) and 10f). On FIG. 10e), white lines corresponding to DNA strands were observed on the receiving coverslip, and besides, on FIG. 10f) exosomes were observed directly on the stamp (highlining by white circle).

Conclusion

The biomarkers can be observed after only one assembly thanks to SEM inspection. The inventors made it also possible to observe them in fluorescence
with both remaining on the stamp or
by removing one on another support for observation and observing the remaining one on the stamp.

1-12. (canceled)

13. A method for in vitro isolating molecules and/or molecular complexes having a radius of gyration smaller or equal to 2 μm from a complex fluid, said method comprising the following steps:
a) contacting a complex fluid with a structured capture array having topographical features in the form of a plurality of plane surfaces in-between cavities, wherein the structured capture array is surrounded by humid air and wherein said complex fluid is a non-Newtonian fluid,
b) covering the deposited complex fluid with a covering means such that the complex fluid is surrounded by a meniscus which comprises a rear meniscus and a front meniscus, wherein the surface tension of the complex fluid between the covering means and the structured capture array defines at least the front and the rear meniscus; and
c) dragging either the covering means or the structured capture array in one direction at a speed of at most 2 mm·s⁻¹ for displacing the complex fluid, wherein the front and the rear menisci are displaced on and along the topographical features of said structured capture array toward said direction, wherein the front meniscus covers uncovered topographical features and the rear meniscus uncovers covered topographical features during displacement of the complex fluid, resulting in that:
the molecules and/or the molecular complexes are trapped inside the cavities, and possibly elongated on the plane surfaces toward the direction of the dragging, wherein the humid air has a humidity of at least 40% based on the air maximal humidity.

14. The method for in vitro isolating molecules and/or molecular complexes according to claim 13, wherein the humidity in step c) is from 40 to 80% based on the maximal moisture content of the surrounding air.

15. The method for in vitro isolating molecules and/or molecular complexes according to claim 13, wherein the molecules are biological molecules, in particular the biological molecules are nucleic acid molecules, particularly the nucleic acid molecules are selected from the group comprising viral nucleic acid molecules, chromatin, circulating free DNA, RNA, linear DNA, linear RNA, circular DNA, circular RNA, single-stranded DNA, double-stranded DNA, and tumoral DNA.

16. The method for in vitro isolating molecules and/or molecular complexes according to claim 13, wherein the molecular complexes are biological complexes, in particular the biological molecular complexes are selected in the group comprising vacuoles, lysosomes, transport vesicles, secretory vesicles, liposomes, ectosomes, microvesicles, virus, part of virus, exosomes and macro complex.

17. The method for in vitro isolating molecules and/or molecular complexes according to claim 13, wherein the complex fluid is a biological fluid of an individual, in particular said biological fluid is selected in the group consisting of cerebrospinal fluid, pleural effusion, saliva, urine, blood, plasma and serum, especially the biological fluid is blood.

18. The method for in vitro isolating molecules and/or molecular complexes according to claim 13, wherein before or at step a), the complex fluid is blended with a surfactant, in particular a non-ionic surfactant.

19. The method for in vitro isolating molecules and/or molecular complexes according to claim 18, wherein before or at step a), the complex fluid is blended with 0.1 to % v/v Triton X100, particularly 0.3% v/v TritonX100.

20. The method for in vitro isolating molecules and/or molecular complexes according to claim 13, wherein molecules and/or molecular complexes of different radius of gyration have to be isolated, and wherein step c) is carried out at least two times at different speeds for each radius of gyration.

21. The method for in vitro isolating molecules and/or molecular complexes according to claim 13, wherein molecules and/or molecular complexes of different radius of gyration have to be isolated, wherein the structured capture array comprises at least a first and a second portions of topographical features wherein the first portion has larger cavities than the second one, such that step c) results in the spatial separation onto the structured capture array of the isolated molecules and/or molecular complexes of different radii of gyration.

22. The method for in vitro isolating molecules and/or molecular complexes according to claim 13, wherein the cavities of the structured capture array are functionalised with a linking element configured for trapping the molecules and/or complexes.

23. The method for in vitro isolating molecules and/or molecular complexes according to claim 13, wherein the process of the invention comprises a further step d):
d) contacting the surface of the structured capture array with a printing surface for transferring the trapped molecules and/or molecular complexes from the surface of the structured capture array to the printing surface.

24. A structured capture array for in vitro isolating molecules and/or molecular complexes having a radius of gyration smaller than 2 μm from a complex fluid comprising numerous components, wherein the structured capture array having topographical features in the form of a plurality of plane surfaces in-between cavities.
